(19) United States
(12) Patent Application Publication
(10) Pub. No.: US 2026/0091085 A1
(43) Pub. Date: Apr. 2, 2026

(54) COMPOSITION FOR SYSTEMIC ANTICANCER TREATMENT IN LOCALIZED RADIOTHERAPY

(71) Applicant: INJE UNIVERSITY INDUSTRY-ACADEMIC COOPERATION FOUNDATION, Gimhae-si (KR)

(72) Inventors: Sae Gwang PARK, Busan (KR); Jin Hee PARK, Busan (KR)

(73) Assignee: INJE UNIVERSITY INDUSTRY-ACADEMIC COOPERATION FOUNDATION, Gimhae-si (KR)

(21) Appl. No.: 19/317,066

(22) Filed: Sep. 2, 2025

Related U.S. Application Data
(63) Continuation of application No. PCT/KR2024/095461, filed on Feb. 27, 2024.

(30) Foreign Application Priority Data
Mar. 8, 2023 (KR) 10-2023-0030558
Feb. 22, 2024 (KR) 10-2024-0025693

(51) Int. Cl. A61K 38/21; A61N 5/10; A61P 35/00; C07K 16/28

(52) U.S. Cl. CPC A61K 38/215; A61N 5/10; A61P 35/00; C07K 16/2818; C07K 16/2827; A61N 2005/1092

(57) ABSTRACT

The present invention relates to a composition for the prevention or treatment of metastatic cancer, capable of inducing an abscopal effect, which is a systemic anticancer effect, in combination with radiotherapy to treat metastatic cancer that has spread from a primary cancer, wherein, by having confirmed that, upon radiotherapy in which a primary cancer site was irradiated with a low dose of radiation, the abscopal effect, which is a systemic anticancer effect, did not appear, whereas, when type 1 interferon was administered in combination with low-dose radiation to the primary cancer site, an anticancer effect appears even in non-radiated metastatic cancer sites, a composition comprising type 1 interferon is provided as a therapeutic agent or adjuvant for the treatment of metastatic cancer, which induces a systemic anticancer effect upon low-dose radiotherapy.

### CROSS-REFERENCE TO RELATED APPLICATION

This application is a Continuation of PCT International Patent Application No. PCT/KR2024/095461 filed on Feb. 27, 2024, which claims priority to Korean Patent Application Nos. 10-2023-0030558 filed on Mar. 8, 2023 and 10-2024-0025693 filed on Feb. 22, 2024 which are all hereby incorporated by reference in their entirety.

### SEQUENCE LISTING

This application contains a Sequence Listing submitted via USPTO Patent Center and hereby incorporated by reference in its entirety. The Sequence Listing is named \2280-603.xml, created on Aug. 31, 2025, and 3,661 bytes in size.

### BACKGROUND OF THE INVENTION

The present disclosure relates to a composition for systemic anticancer treatment in localized radiotherapy, and to prevention or treatment of a metastatic cancer, capable of inducing an abscopal effect, which is a systemic anticancer effect, in combination with radiotherapy to treat a metastatic cancer that is metastasized from a primary cancer.

### BACKGROUND ART

A cancer, a disease that occurs when normal cells undergo changes due to genetic mutations by various causes, is one of the diseases with a high mortality rate in modern times. When cancer develops, cancer cells, which have mutations without following the differentiation, proliferation, growth, and cell cycles of normal cells, proliferate abnormally and are called a malignant tumor when these abnormal cells form a mass. Cancer cells that form malignant tumors may infiltrate tissues surrounding the area where the cancer has developed, and in severe cases, they travel through blood vessels or lymphatic vessels to metastasize to other organs in the body.

Cancer metastasis is a phenomenon that takes place during progression or treatment of the cancer, and even if the primary cancer originating from a site of cancer development is removed or treated, the prognosis is very poor or may lead to death since the possibility of systemic cancer recurrence increases once cancer metastasis takes place. With progression of a primary cancer, it is considered that the rate of metastasis to surrounding lymph nodes and other tissues increases as the tumor size increases, but there are cases where cancer metastasizes despite the cancer being small. In the process of treating cancer, not all cancer treatment methods simultaneously suppress cancer proliferation and metastasis, so even if primary cancer is treated, a metastatic cancer often occur. Therefore, in order to prevent cancer recurrence and improve the prognosis of cancer treatment, development of an effective method capable of preventing or treating development of a metastatic cancer along with cancer treatment is required.

Cancer treatment methods include a tumor removal therapy, which involves surgical elimination of a cancerous area; a chemotherapy which is to administrate anticancer drugs locally or systemically; and a radiotherapy to eliminate cancer by emitting radiation locally or systemically. Tumor removal therapy by surgery may result in the cancer recurring or metastasizing if the cancer cells are not completely removed. In addition, in the case of chemotherapy, which is a method of removing cancer cells by administering anticancer drugs that are generally cytotoxic, there is a concern that side effects may occur due to damages not only cancer cells but also normal cells.

In particular, the radiotherapy for cancer treatment is being used in more than 50% of all solid cancer patients with the development of devices. Radiotherapy is a method of destroying tumors by irradiating cancer cells with radiation to cause DNA damage. It is known that radiation-induced cell death may induce immune response activation through the production and release of immunogenic factors, thereby inducing immunogenic death of cancer cells.

The abscopal effect, one of the effects of radiotherapy, is a systemic anticancer effect of radiotherapy, which is a phenomenon in which treatment effects appear in non-irradiated metastatic tumors after radiotherapy in patients with distant metastases. Preclinical studies have shown that the abscopal effect is dependent on the immune response, and the abscopal effect, a systemic anticancer effect, is induced when high-dose radiation of 6 Gy or more is irradiated at a single dose, with a report that the abscopal effect does not appear at low-dose fractionated radiation that is able to minimize cell damage during radiotherapy.

The inventors of the present disclosure have sought to devise a method of inducing the abscopal effect, a systemic anticancer effect, even during low-dose level radiotherapy capable of minimizing cell damage and identify key molecular factors that determine the presence of the abscopal effect according to the radiation dose, so as to provide a method of treating a metastatic cancer, capable of inducing systemic anticancer treatment during localized radiotherapy by utilizing the same.

### SUMMARY OF THE INVENTION

The present disclosure relates to a composition for preventing or treating a metastatic cancer, capable of inducing an abscopal effect, which is a systemic anticancer effect, in combination with radiotherapy to treat a metastatic cancer that is metastasized from a primary cancer, wherein it was determined in radiotherapy where low-dose radiation is applied to a primary cancer site that no abscopal effect, which is a systemic anticancer effect, was shown, but the anticancer effect was observed even in an unirradiated metastatic cancer site when type 1 interferon is administered in combination with low-dose radiation to the primary tumor site, such that a composition including a type 1 interferon is provided as a therapeutic agent or adjuvant for treating a metastatic cancer that induces a systemic anticancer effect in a low-dose radiotherapy.

The present disclosure provides a pharmaceutical composition for preventing or treating a metastatic cancer that is metastasized from a primary cancer, including a type 1 interferon as an active ingredient.

In addition, the present disclosure provides an adjuvant for radiotherapy for treatment of a metastatic cancer that is metastasized from a primary cancer, including a type 1 interferon as an active ingredient.

According to the present disclosure, although low-dose irradiation to a primary cancer site did not induce an abscopal effect, which is a systemic anticancer effect, during radiotherapy, when a type 1 interferon is administered in combination with application of low-dose radiation to a primary cancer site, the anticancer effect was observed even in a metastatic cancer site that has not been irradiated, such that a composition including the type 1 interferon may be provided as a therapeutic agent or adjuvant for treatment of a metastatic cancer that induces a systemic anticancer effect in low-dose radiotherapy.

### BRIEF DESCRIPTION OF THE DRAWINGS

FIGS. 1A-1C show evaluations on whether an abscopal effect is derived according to a dose of therapeutic radiation in an individual with multiple tumors with metastases progressed, wherein FIG. 1A is a diagram illustrating a method of irradiating primary and secondary tumors formed in an individual with multiple tumors, FIG. 1B is a graph showing changes in a size of a primary tumor formed subcutaneously on the back of the irradiated head side after irradiation, and FIG. 1C is a graph showing changes in a size of a secondary tumor formed subcutaneously on the back of the unirradiated tail side after irradiation. The red arrow indicates a starting point of irradiation.

FIGS. 2A-2D show analyses from a molecular perspective on whether an abscopal effect is present following irradiation in an individual with multiple tumors with advanced metastasis, wherein FIG. 2A shows a result of evaluating changes in an expression level of an IFN-β protein according to the type of radiation exposure, FIG. 2B shows a result of evaluating changes in an expression level of a cGAS protein according to the type of radiation exposure, FIG. 2C shows a result of evaluating changes in an expression level of a STING protein according to the type of radiation exposure, and FIG. 2D shows results of evaluating changes in an expression level of a type I interferon (IFN) gene according to the type of radiation exposure.

FIGS. 3A-3F show results of evaluating whether an abscopal effect, a systemic anticancer effect, is induced upon low-dose irradiation following administration of type 1 interferon in breast cancer, lung cancer, and colon cancer models. FIG. 3A is a graph showing changes in a size of a primary tumor formed subcutaneously on the back of the irradiated head side following administration of a type 1 interferon in a breast cancer model, and FIG. 3B is a graph showing changes in a size of a secondary tumor formed subcutaneously on the back of the unirradiated tail side following administration of a type 1 interferon in a breast cancer model. FIG. 3C is a graph showing changes in a size of a primary tumor formed subcutaneously on the back of the irradiated head side following administration of a type 1 interferon in a lung cancer model, and FIG. 3D is a graph showing changes in a size of a secondary tumor formed subcutaneously on the back of the unirradiated tail side following administration of a type 1 interferon in a lung cancer model. FIG. 3E is a graph showing changes in a size of a primary tumor formed subcutaneously on the back of the irradiated head side following administration of a type 1 interferon in a colon cancer model, and FIG. 3F is a graph showing changes in a size of a secondary tumor formed subcutaneously on the back of the unirradiated tail side following administration of a type 1 interferon in a colon cancer model. The red arrow indicates a starting point of irradiation.

### DETAILED DESCRIPTION OF THE INVENTION

The terms used in the present specification have been selected from general terms that are currently widely used as much as possible while considering the functions in the present disclosure, but these may vary depending on the intention of those skilled in the art or precedents or the emergence of new technologies. Additionally, in certain cases, there are terms arbitrarily selected by the applicant, and in such cases, meanings thereof will be set forth in detail in the detailed description of the disclosure. Therefore, the terms used herein should be defined based on the meaning of the terms and the overall content of the present disclosure, rather than simply the names of the terms.

Unless otherwise defined, all terms used herein, including technical or scientific terms, have the same meaning as commonly understood by those skilled in the art to which the present disclosure pertains. Terms defined in commonly used dictionaries should be interpreted as having a meaning consistent with the meaning in the context of the relevant art, but not be interpreted in an idealized or overly formal sense, unless explicitly defined in this application.

The numerical range includes the numerical values defined in the above range. All maximum numerical limits given throughout the specification include all lower numerical limits as if the lower numerical limits were expressly written. All minimum numerical limits given throughout this specification include all higher numerical limits as if the higher numerical limits were expressly written. All numerical limits given throughout this specification will include all the better numerical range within the broader numerical range, as if the narrower numerical limits were explicitly written.

Hereinafter, the present disclosure will be described in more detail.

The present disclosure provides a pharmaceutical composition for preventing or treating a metastatic cancer that is metastasized from a primary cancer, including a type 1 interferon as an active ingredient.

The type I interferon is a multi-gene cytokine family that includes IFN-α with 14 subtypes and IFN-β, a single type, as well as IFN-ε, IFN-τ, IFN-κ, IFN-ω, IFN-δ, and IFN-ζ, whose roles are unknown. The type 1 interferon has a mechanism in which it recognizes cytosolic DNA caused by DNA damage or cell death and is increased by cGAS/STING signaling, with a report that the expression of type 1 interferon increases upon irradiation.

Preferably, in the present disclosure, the type 1 interferon may be IFN-α or IFN-β, and specifically, it may be a recombinant IFN-β (rhIFN-β) consisting of an amino acid sequence represented by SEQ ID NO: 3.

The primary cancer is a cancer formed by a tumor originating from a site where cancer has developed, and the primary cancer herein is any one or more cancers selected from the group consisting of brain cancer, oral cancer, laryngeal cancer, pharyngeal cancer, stomach cancer, lymphoma, thyroid cancer, esophageal cancer, lung cancer, breast cancer, gallbladder cancer, liver cancer, kidney cancer, pancreatic cancer, prostate cancer, ovarian cancer, bladder cancer, cervical cancer, small intestine cancer, colon cancer, rectal cancer, colorectal cancer, bone cancer, skin cancer, endocrine cancer, and blood cancer.

The metastatic cancer occurs in an area other than the part where the primary cancer is developed by cancer cells of the primary cancer via blood vessels or lymphatic vessels, and herein it refers to all cancers that may be developed by metastasis by the primary cancer and may include microscopic tumors that are difficult to diagnose.

The pharmaceutical composition is administered after radiotherapy for cancer treatment, and the radiotherapy may include localized irradiation of the primary cancer site with ablative or non-ablative low-dose radiation at a level of greater than 0 Gy and up to 6 Gy per fraction.

The pharmaceutical composition may further include an immune checkpoint inhibitor.

The immune checkpoint inhibitor is a type of drug that blocks proteins called immune checkpoints expressed on some immune cells, such as T cells, and cancer cells, as a form of immunotherapy for treating cancer.

In the present disclosure, the immune checkpoint inhibitor may be one or more selected from the group consisting of anti-CTLA4 antibody, anti-PD-L1 antibody, and anti-PD-1 antibody.

The anti-CTLA4 antibody is an antibody targeting cytotoxic T lymphocyte antigen-4 (CTLA4), a derivative thereof, or an antigen-binding fragment thereof, the anti-PD-L1 antibody is an antibody targeting programmed death ligand 1 (PD-L1), a derivative thereof, or an antigen-binding fragment thereof, and the anti-PD-1 antibody is an antibody targeting programmed death 1 (PD-1), a derivative thereof, or an antigen-binding fragment thereof.

Specific immune checkpoint inhibitors used may include, but are not limited to, ipilimumab and tremelimumab targeting CTLA4; atezolizumab, avelumab, and durvalumab targeting PD-L1; pembrolizumab, nivolumab, dostarlimab, retifanlimab-dlwr, tislelizumab and cemiplimab targeting PD-1; and relatlimab targeting LAG-3.

The immune checkpoint inhibitor may induce an abscopal effect, a systemic anticancer effect, by being administered in combination with radiotherapy.

The pharmaceutical composition of the present disclosure may be manufactured in the form of a unit dose by formulating using a carrier that is pharmaceutically acceptable or manufactured by encapsulating in a large-capacity container, in accordance with a method that may be easily carried out by a person with ordinary skill in the art to which the disclosure pertains.

The pharmaceutically acceptable carriers are those that are commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additive included in the pharmaceutical composition is not particularly limited and may be appropriately adjusted within a content range used in the conventional preparation.

The pharmaceutical composition may be formulated in the form of one or more external agents selected from the group consisting of injectable formulations such as aqueous solutions, suspensions, and emulsions, pills, capsules, granules, tablets, creams, gels, patches, sprays, ointments, plasters, lotions, liniments, pastas, and cataplasmas.

The pharmaceutical composition may include a pharmaceutically acceptable carrier and diluent that are additionally present for formulation. The pharmaceutically acceptable carriers and diluents include, but are not limited to, excipients such as starch, sugars, and mannitol, fillers and extenders such as calcium phosphate, cellulose derivatives such as carboxymethylcellulose and hydroxypropyl cellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, and elixirs. When administered parenterally, it may be formulated as injection solutions, suppositories, powders for respiratory inhalation, aerosols for sprays, ointments, powders for application, oils, and creams.

The dosage range of the pharmaceutical composition of the present disclosure may vary depending on the patient's condition, weight, age, sex, health status, dietary constitution specificity, nature of the preparation, severity of a disease, administration time for composition, method of administration, duration or interval of administration, excretion rate, and drug form, and it may be appropriately selected by a person skilled in the art. For example, it may range from about 0.1 to 10,000 mg/kg, but is not limited thereby, and it may be administered once to several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, mode of administration, administration time, and/or administration route, and a person with ordinary skill in the art may easily determine and prescribe the effective dosage for the desired treatment. The administration of the pharmaceutical composition of the present disclosure may be conducted once a day or in several divided doses.

Furthermore, the present disclosure provides an adjuvant for radiotherapy for treatment of a metastatic cancer that is metastasized from a primary cancer, including a type 1 interferon as an active ingredient.

The radiotherapy may include localized irradiation of a primary cancer site with ablative or non-ablative low-dose fractionated radiation at a level of greater than 0 Gy and up to 6 Gy per fraction, and the adjuvant may be administered within one month immediately after the radiotherapy.

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### Example 1. Evaluation on the Presence of an Abscopal Effect Following Radiotherapy in an Individual with Multiple Tumors

Evaluation was conducted on whether the abscopal effect appears depending on a dose of therapeutic radiation in an individual with multiple tumors with advanced metastases.

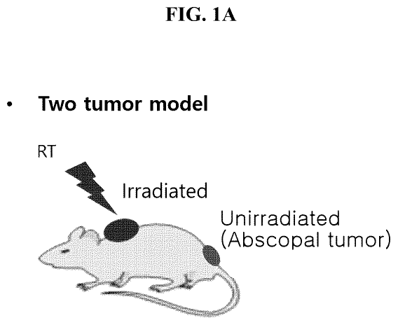

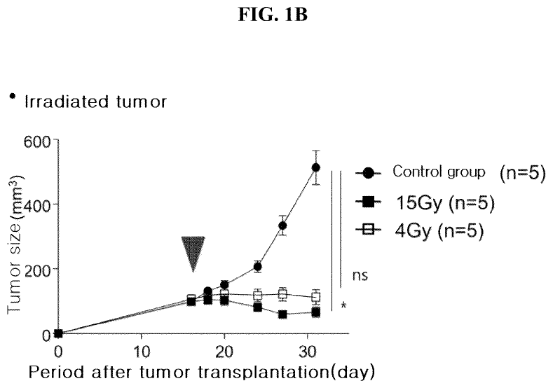

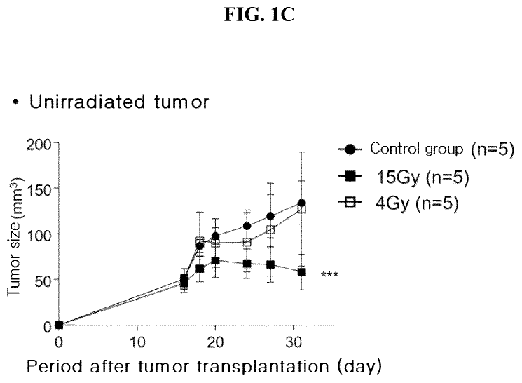

TUBO cells, which are rodent-derived breast cancer cells, were suspended in phosphate buffered saline (PBS) at a dose of 1×10⁶ cells/200 μl. TUBO cells were injected subcutaneously into the back of 6- to 8-week-old Balb/c mice, first into the head and then into the tail, and the tumor sizes were measured at intervals of 2 to 3 days (FIG. 1A). When the tumor size reached an average of 120 mm3 (width×length×height/2), radiation was applied. Depending on the biologically effective dose (BED) value (BED=nd [1+d/(α/β)], n; number of fractions, d; fractionated radiation dose) of radiation applied to the primary tumor formed subcutaneously on the back of the head side, 15 Gy (gray) of radiation was irradiated as an ablative dose at a similar level in a single fraction (BED value=90.3), or 40 Gy of radiation was irradiated at a non-ablative dose in 10 fractions (4 Gy/fraction 10 times, BED value=93.3). Areas other than the primary tumor formed subcutaneously on the back of the head side were shielded to make the radiation only directed to the primary tumor area. After irradiation, by measuring the size of the primary tumor formed subcutaneously on the back of the head side and the secondary tumor formed subcutaneously on the back of the tail side, evaluation was made on whether the abscopal effect appears following irradiation.

As shown in FIG. 1B, the size of the primary tumor formed subcutaneously on the back of the irradiated head side was found to be significantly reduced in both cases of irradiation with 15 Gy at an ablative dose and 4 Gy/fraction 10 times at a non-ablative dose. However, as shown in FIG. 1C, the size of the secondary tumor formed subcutaneously on the back of the tail side with no irradiation decreased only when irradiated with 15 Gy of radiation at an ablative dose, and when irradiated with 4 Gy/fraction of radiation at a non-ablative dose, the size of the tumor was similar to that of a control group that did not receive radiotherapy. The results demonstrate that the abscopal effect, which is a systemic anticancer treatment effect, does not occur when 4 Gy/fraction of radiation was irradiated at a non-ablative dose.

### Example 2. Assessment of Changes in Signaling Systems at a Molecular Level Depending on the Type of Radiation Exposure

### 2-1. Changes in Expression Levels of IFN-β Proteins

To analyze the presence of the abscopal effect following irradiation from a molecular perspective, changes in IFN-β expression depending on the type of radiation exposure were evaluated.

According to the method described in Example 1 above, an individual with multiple tumors was irradiated, and 48 hours later, the irradiated tumor area was separated and weighed. Tumors were suspended in 100 μl of phosphate buffer saline (PBS) containing protease inhibitors 4-(2-aminoethyl)benzenesulfonyl fluoride (AEBSF), aprotinin, bestatin, aloxistatin (E-64), leupeptin, pepstatin A, and dimethyl sulfoxide (DMSO) per 0.1 g of tumor, and dissociated into single cells using a cell dissociator (gentleMACS dissociator; Miltenyi biotec). The separated cells were centrifuged at 1,500 rpm for 5 minutes, only the supernatant was collected, and the concentration of IFN-β contained in the supernatant was measured using bicinchoninic acid (BCA) reagent. Samples, standards, or controls whose protein concentrations were measured were added at 50 μl per well to a 96-well plate coated with IFN-β anti-tag monoclonal antibodies, and 50 μl of anti-IFN-β antibodies were treated and then incubated for 1 hour at room temperature for a reaction. The wells where the reaction was completed were washed with PBS and treated with 100 μl of substrate solution to induce color development for 5 to 15 minutes, and then color development was terminated with a stop solution. The concentration of IFN-β was evaluated by measuring the absorbance at 450 nm using a microplate reader.

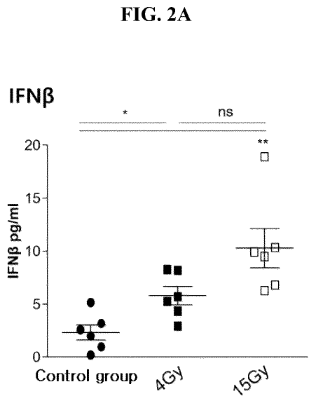

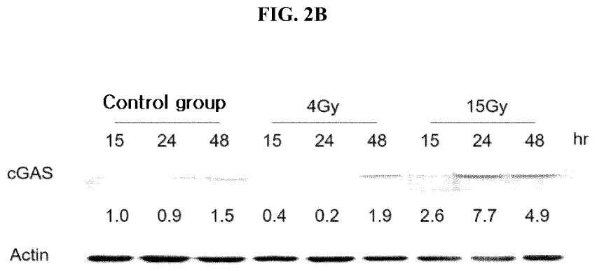

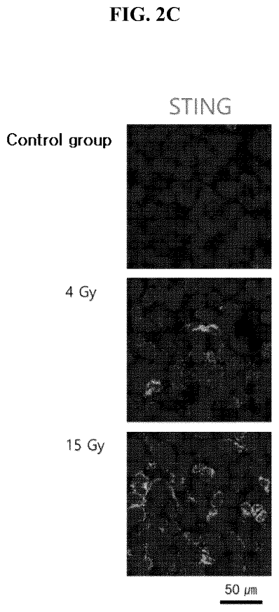

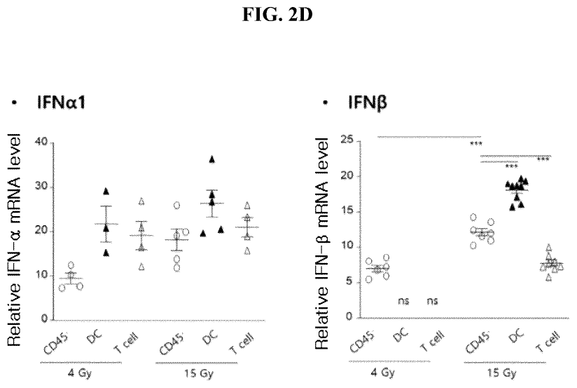

As shown in FIG. 2A, as a result of measuring the concentration of IFN-β in the primary tumor formed subcutaneously on the back of the irradiated head side, the concentration of IFN-β was found to be significantly increased when irradiated with 15 Gy of radiation at an ablative dose that showed the abscopal effect, compared to when irradiated with 4 Gy/fraction of radiation at a non-ablative dose.

2-2. Changes in Expression Levels of cGAS Proteins

To analyze the presence of the abscopal effect following radiation exposure from a molecular perspective, evaluation was conducted on changes in the expression of cGAS, an upstream signaling system of type I IFN, according to the type of radiation exposure.

According to the method described in Example 1, an individual with multiple tumors was irradiated, and 15 hours, 24 hours, or 48 hours after irradiation, the tumors were isolated and suspended in RIPA buffer containing the same protease inhibitor as in 2-1 above at a level of 100 μl per 0.1 g of tumor for lysis of the tumors at a cellular level. After incubation for 2 hours, centrifugation was performed at 13,000 rpm for 15 minutes, and only the supernatant was collected to measure the concentration of cGAS protein. The sample was placed in a sample buffer containing sodium dodecyl sulfate (SDS), heated at 95° C. to 100° C. for 10 minutes, and transferred to a polyacrylamide-SDS gel to carry out electrophoresis. The gel with the electrophoresis completed was transferred to a nitrocellulose membrane, and protein expression levels were measured using anti-cGAS and anti-actin antibodies.

As shown in FIG. 2B, as a result of measuring the concentration of cGAS in the primary tumor formed subcutaneously on the back of the irradiated head side, the concentration of cGAS was found to be increased significantly when irradiated with 15 Gy of radiation at an ablative dose that showed the abscopal effect compared to when irradiated with 4 Gy/fraction of radiation at a non-ablative dose.

### 2-3. Changes in Expression Levels of STING Proteins

To analyze the presence of the abscopal effect following radiation exposure from a molecular perspective, evaluation was conducted on changes in the expression of STING, an upstream signaling system of type I IFN, according to the type of radiation exposure.

An individual with multiple tumors was irradiated according to the method described in Example 1 above, and the tumors were isolated 48 hours after irradiation. Isolated tumors were fixed in 10% formalin for 24 hours. Paraffin blocks were formed using a tissue processor (Leica), and tissues were sectioned at 4 μm thickness using a sectioning machine (Leica) and mounted on slides. The tissue attached to the slide was subjected to de-paraffinization, re-hydration, and antigen retrieval, reacted with anti-STING antibody, and labeled with secondary fluorescent antibody (green), and then the cell nuclei was stained with 4′,6-diamidino-2-phenylindole (DAPI) (blue), followed by observation under a light microscope.

As shown in FIG. 2C, as a result of measuring the expression level of STING in the primary tumor formed subcutaneously on the back of the irradiated head side, the expression level of STING was found to significantly increase when irradiated with 15 Gy of radiation at an ablative dose that showed the abscopal effect, compared to when irradiated with 4 Gy/fraction of radiation at a non-ablative dose.

### 2-4. Changes in Expression Levels of Type 1 Interferon Genes at a Single Cell Level

To analyze the presence of the abscopal effect following radiation exposure from a molecular perspective, the expression level of type I IFN genes depending on the type of radiation exposure was evaluated at a single-cell level.

An individual with multiple tumors was irradiated according to the method described in Example 1 above, and tumor tissues were isolated 48 hours after irradiation. Tumor tissues were suspended in phosphate buffer saline (PBS) containing collagenase (1.5 mg/ml) and DNase (0.4 mg/ml) and dissociated into single cells using a cell collection device (gentleMACS dissociator; Miltenyi biotec). Expression factors on the cell surface were labeled with anti-CD45.2-APC, anti-CD11c-FITC, and anti-CD8-PE antibodies. Cells labeled with expression factors on the cell surface were separated into CD45-cells, dendritic cells (CD45⁺CD11c⁺), or CD8 T cells (CD45⁺CD8+) using the Aria (BD) cell sorter. RNA was isolated from each cell using TRI reagent (Invitrogen), and the expression levels of IFN-α (forward: SEQ ID NO: 1, reverse: SEQ ID NO: 2) or IFN-β (BioRad, ID: qSscCED0010658) were measured by real-time PCR.

As shown in FIG. 2D, as a result of measuring the expression level of type I IFN gene in the primary tumor formed subcutaneously on the back of the irradiated head side, the expression levels of IFN-α and IFN-β were significantly increased all in CD45-cells, dendritic cells (CD45⁺CD11c⁺), and CD8 T cells (CD45⁺CD8+) when irradiated at an ablative dose of Gy that produced the abscopal effect. On the other hand, when 4 Gy/fraction of radiation was applied at a non-ablative dose, it was found that IFN-β expression was not observed in dendritic cells (CD45⁺CD11c⁺) and CD8 T cells (CD45⁺CD8+).

The above results demonstrate that the molecular factors determining the abscopal effect, which is a systemic anticancer effect following radiation exposure, are affected by the expression levels of IFN-β, cGAS, and STING at the protein level, and by the expression levels of IFN-β genes in dendritic cells (CD45⁺CD11c⁺) and CD8 T cells (CD45⁺CD8+) at the gene level. Therefore, when low-dose radiotherapy is carried out to reduce tissue destruction by radiation in individuals with multiple tumors with advanced metastases, it was revealed that IFN-β, cGAS, and STING may be key factors to induce the abscopal effect, which exhibits a systemic anticancer effect.

### Example 3. Evaluation on Whether the Abscopal Effect is Induced Depending on the Type 1 Interferon Treatment

To evaluate whether low-dose irradiation induces the abscopal effect, a systemic anticancer effect, following administration of type I interferon, treatment was followed by administration of the type I interferon in combination with irradiation to individuals with multiple tumors. Breast cancer (TUBO), lung cancer (TC-1), and colon cancer (CT-26) cells were used to create breast cancer, lung cancer, and colon cancer tumor models. With the same TUBO cells as in Example 1, TC-1 cells were suspended in PBS at a concentration of 2×10⁶ cells/200 μl, and CT-26 cells were suspended in PBS at a concentration of 1×10⁶ cells/200 μl. For breast cancer cells, TUBO cells were injected subcutaneously into the backs of 6- to 8-week-old Balb/c mice, first into the head side and then into the tail side, using the same method as in Example 1. For lung cancer cells, TC-1 cells were injected subcutaneously into the back of 6- to 8-week-old C57bl/6 mice, first into the head side, and then into the tail side. For colon cancer, CT-26 cells were injected subcutaneously first into the head side and then into the tail side of 6- to 8-week-old Balb/c mice, respectively. Tumor size was measured at 2-3 day intervals in mice injected with each cancer cell to form tumors.

According to the method described in Example 1 above, 4 Gy/fraction of radiation was applied to an individual with multiple tumors at a non-ablative dose, and 48 hours after irradiation, 0.1 μg/10 μl recombinant IFN-β (rhIFN-β, Peprotech, represented by amino acids of SEQ ID NO: 3) was directly injected into the primary tumor site formed subcutaneously on the back on the irradiated head side. Seven days after radiation exposure, anti-PD-L1 antibody (BioXcell, 10F.9G2), an immune checkpoint inhibitor, was administered intraperitoneally at a dose of 5 mg/kg. By measuring the size of the primary tumor formed subcutaneously on the back of the head side and the secondary tumor formed subcutaneously on back of the tail side, evaluation was conducted on whether the abscopal effect occurred following the administration of type 1 interferon.

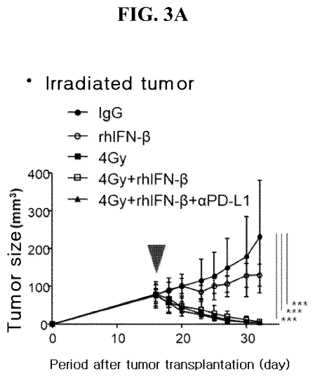

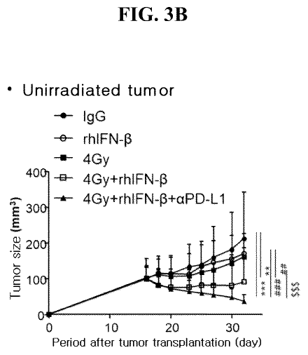

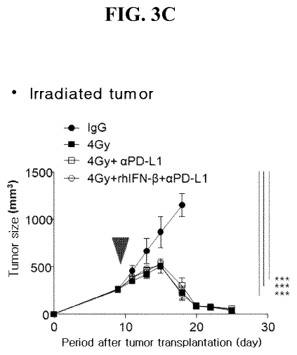

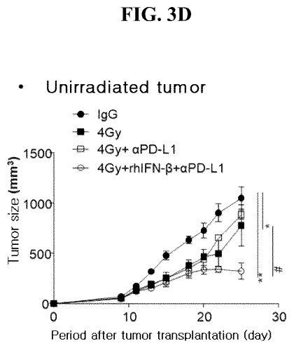

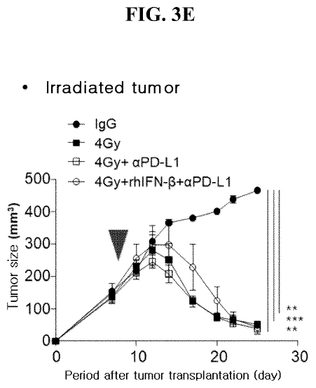

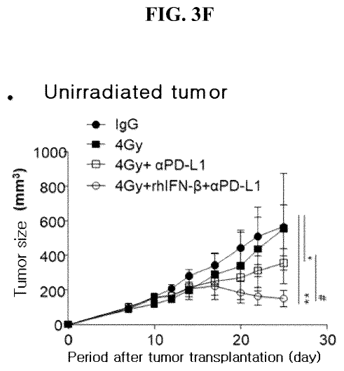

In the case of the breast cancer model, as shown in FIG. 3A, when 4 Gy/fraction of radiation was applied at a non-ablative dose to the primary tumor formed subcutaneously on the back of the irradiated head side, the tumor size was reduced. On the other hand, when only rhIFN-β was administered, the tumor size was similar to that of the control group, and it was significantly reduced when rhIFN-β was administered together with radiation exposure or rhIFN-β and αPD-L1 were administered, Ultimately, it was proven that the tumor area that received radiotherapy showed anticancer effects only when irradiated, but rhIFN-β administration alone did not derive the anticancer effect. In contrast, as shown in FIG. 3B, in the secondary tumors formed subcutaneously on the back of the unirradiated tail side, the tumor size was maintained similar to that of the control group when the primary tumor site was subjected to the irradiation only or rhIFN-β administration alone. However, when rhIFN-β was administered together with radiation to the primary tumor site, the size of the secondary tumor significantly decreased, and when rhIFN-β and αPD-L1 were administered, the tumor size was significantly reduced.

The above results demonstrate that rhIFN-β administered in combination with radiotherapy to the primary tumor site derives an abscopal effect showing the antitumor effect even to the secondary tumors that have not undergone radiotherapy that provides the same environment as the metastatic cancer that is metastasized from a primary cancer. Referring to the results of the breast cancer model above, where rhIFN-β administration alone did not show an antitumor effect, experiments were set up in lung cancer models and colon cancer models with single administration of the immune checkpoint inhibitor anti-PD-L1 antibody (αPD-L1) and co-administration of αPD-L1 and rhIFN-β to evaluate changes in the antitumor effect on the metastatic cancer.

In the case of the lung cancer model, as shown in FIG. 3C, similar to the breast cancer model, the tumor size decreased upon irradiation in the primary tumor site where radiotherapy was performed. On the other hand, as shown in FIG. 3D, in the secondary tumor site with no radiation, the tumor size was maintained similar to that of the control group even when radiotherapy was performed on the primary tumor site or αPD-L1 was administered together with radiotherapy, but the size of the secondary tumor was found to be significantly reduced when rhIFN-β was co-administered with radiotherapy to the primary tumor site.

In addition, in the case of the colon cancer model, as shown in FIG. 3E, similar to the breast cancer model, the tumor size decreased upon irradiation in the primary tumor site where radiotherapy was performed. In contrast, as shown in FIG. 3F, in the secondary tumor site that was not irradiated, the tumor size was maintained at a similar size to the control group when radiotherapy was performed on the primary tumor site, and when αPD-L1 was co-administered with radiotherapy, the tumor size decreased slightly, but the size of the secondary tumor was significantly reduced when the primary tumor site was subjected to irradiation along with rhIFN-β administration.

The above results demonstrate that the abscopal effect, a systemic anticancer effect, was not observed in breast, lung, and colon cancer models when non-ablative low-dose radiotherapy was conducted at a level of 4 Gy/fraction, but when radiotherapy was co-applied with type 1 interferon administration, the abscopal effect, a systemic anticancer effect, was induced even in non-ablative low-dose radiotherapy at a level of 4 Gy/fraction. Furthermore, it was demonstrated that the abscopal effect, a systemic antitumor effect, was induced even with non-ablative low-dose radiotherapy at a level of 4 Gy/fraction when type I interferon and immune checkpoint inhibitors were administered along with radiotherapy.

Therefore, the above results demonstrate that a composition including the type 1 interferon may be used as a therapeutic agent or adjuvant for the treatment of a metastatic cancer, capable of inducing a systemic anticancer effect during low-dose radiotherapy.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

What is claimed is:

1. A method of preventing or treating a metastatic cancer that is metastasized from a primary cancer in a subject in need thereof, comprising:
administering a pharmaceutical composition comprising a type 1 interferon as an active ingredient to the subject.

2. The method of claim 1, wherein the type 1 interferon is IFN-α or IFN-β.

3. The method of claim 1, wherein the type 1 interferon consists of an amino acid sequence represented by SEQ ID NO: 3.

4. The method of claim 1, wherein the primary cancer is any one or more cancers selected from the group consisting of brain cancer, oral cancer, laryngeal cancer, pharyngeal cancer, stomach cancer, lymphoma, thyroid cancer, esophageal cancer, lung cancer, breast cancer, gallbladder cancer, liver cancer, kidney cancer, pancreatic cancer, prostate cancer, ovarian cancer, bladder cancer, cervical cancer, small intestine cancer, colon cancer, rectal cancer, colorectal cancer, bone cancer, skin cancer, endocrine cancer, and blood cancer.

5. The method of claim 1, wherein the pharmaceutical composition is administered after radiotherapy for cancer treatment.

6. The method of claim 5, wherein the radiotherapy comprises localized irradiation of a primary cancer site with ablative or non-ablative low-dose radiation at a level of greater than 0 Gy and up to 6 Gy per fraction.

7. The method of claim 1, wherein the pharmaceutical composition further comprises an immune checkpoint inhibitor.

8. The method of claim 7, wherein the immune checkpoint inhibitor is one or more selected from the group consisting of anti-CTLA4 antibody, anti-PD-L1 antibody, and anti-PD-1 antibody.

9. A method for enhancing an efficacy of radiotherapy for treatment a metastatic cancer that is metastasized from a primary cancer in a subject, comprising:
administering an adjuvant comprising a type 1 interferon as an active ingredient to the subject in conjunction with the radiotherapy.

10. The method of claim 9, wherein the type 1 interferon is IFN-α or IFN-β.

11. The method of claim 9, wherein the type 1 interferon consists of an amino acid sequence represented by SEQ ID NO: 3.

12. The method of claim 9, wherein the radiotherapy comprises localized irradiation of a primary cancer site with ablative or non-ablative low-dose radiation at a level of greater than 0 Gy and up to 6 Gy per fraction.

13. The method of claim 9, wherein the adjuvant is administered within one month immediately after the radiotherapy.

14. The method of claim 9, wherein the primary cancer is any one or more cancers selected from the group consisting of brain cancer, oral cancer, laryngeal cancer, pharyngeal cancer, stomach cancer, lymphoma, thyroid cancer, esophageal cancer, lung cancer, breast cancer, gallbladder cancer, liver cancer, kidney cancer, pancreatic cancer, prostate cancer, ovarian cancer, bladder cancer, cervical cancer, small intestine cancer, colon cancer, rectal cancer, colorectal cancer, bone cancer, skin cancer, endocrine cancer, and blood cancer.

15. The method of claim 9, wherein the adjuvant further comprises an immune checkpoint inhibitor.

16. The method of claim 15, wherein the immune checkpoint inhibitor is one or more selected from the group consisting of anti-CTLA4 antibody, anti-PD-L1 antibody, anti-PD-1, and anti-LAG-3 antibody.


SEQUENCE LISTING (3 sequences)

SEQ ID NO: 1 - DNA, 18 bp, synthetic construct
  source 1..18: mol_type = other DNA
ttgaaggtcctggcacag

SEQ ID NO: 2 - DNA, 19 bp, synthetic construct
  source 1..19: mol_type = other DNA
gaggttcaaggtctgctga

SEQ ID NO: 3 - protein, 166 aa, synthetic construct
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN